(19) United States
(12) Patent Application Publication
(10) Pub. No.: US 2023/0390089 A1
(43) Pub. Date: Dec. 7, 2023

(54) IMPLANT

(71) Applicant: SENTRONIC INTERNATIONAL CORP., New Taipei City (TW)

(72) Inventor: TUNG-KUO TSAI, New Taipei City (TW)

(21) Appl. No.: 17/884,133

(22) Filed: Aug. 9, 2022

(30) Foreign Application Priority Data
Jun. 6, 2022 (TW) 111120886

(51) Int. Cl. A61F 2/90; A61C 8/00

(52) U.S. Cl. CPC A61F 2/90; A61C 8/0037; A61C 2008/0046; A61F 2230/0023; A61F 2250/0067

(57) ABSTRACT

An implant is provided and has an outer surface and multiple cavities. Multiple openings are formed on the outer surface and the cavities communicate with the openings. The maximum latitudinal section area of each cavity is larger than the area of the opening. The latitudinal section of the cavity is parallel with the outer surface of the implant. Therefore, the openings formed on the implant communicate with the cavities, such that the medicine applied on the implant can flow into the cavities. Further, because the maximum latitudinal section of each cavity is larger than the corresponding opening, after flowing into the cavities, the medicine would not easily flow out via the openings.

### BACKGROUND OF THE INVENTION

### 1. Field of the Invention

The present invention relates to an implant that is configured to put into a human body, especially to an implant with surface treatment.

### 2. Description of the Prior Arts

In many surgeries, artificial objects may be put into human bodies, and these artificial objects are so-called implants. One of the common implants is an artificial tooth root for the dental implant surgery, and another one is a stent for cardiac catheterization. The artificial tooth root is configured to be inserted into the gum, so the gum should be drilled and thus form a hole for the artificial tooth root. After a period of time, the bone of the gum may grow and thus the artificial tooth root can be secured on the gum. However, the growth speed of the bone is very slow, which causes inconvenience to the patient after the surgery. Thus, how to improve the growth speed of the bone is an issue of the dental implant surgery. On the contrary, after the cardiac catheterization, if new blood vessels are developed (i.e. angiogenesis), the implanted blood vessel is prone to blockage again. Thus, how to prevent angiogenesis is an issue of the cardiac catheterization.

To overcome the shortcomings, the present invention provides an implant to mitigate or obviate the aforementioned problems.

### SUMMARY OF THE INVENTION

The main objective of the present invention is to provide an implant that is capable of absorbing more medicine, such that the medicine can assist the implanted tissue of the human body in recovery, or prevent the implanted tissue from development.

The implant has an outer surface and a plurality of cavities. The outer surface forms a plurality of openings. The cavities communicates with the openings respectively.

The implant which is a stent has a plurality of wire structures and a plurality of tube structures. The tube structures are formed on each one of the wire structures. Each one of the tube structures forms a cavity, one end of each one of the tube structures is connected to the wire structure, and another end forms an opening communicating with the cavity.

Therefore, because the outer surface of the implant of the present invention forms multiple openings and the openings communicate with the cavities in the implant, the medicine may flow into the cavities after applied on the implant. The latitudinal section of each cavity is larger than the corresponding opening, so after flowing into the cavities, the medicine would not easily flow out, which allows the implant to absorb more medicine. Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an implant in accordance with a first embodiment of the present invention;

FIG. 2 is a sectional view of the implant of the first embodiment in FIG. 1;

FIG. 3 is a sectional view of the implant in another configuration of the first embodiment in FIG. 1;

FIG. 4 is a sectional view of the implant in another configuration of the first embodiment in FIG. 1;

FIG. 5 is a sectional view of the implant in another configuration of the first embodiment in FIG. 1;

FIG. 6 is a perspective view of an implant in accordance with a second embodiment of the present invention;

FIG. 7 is a sectional view of the implant of the second embodiment in FIG. 6;

FIG. 8 is a perspective view of one of wire structures of the implant in another configuration of the second embodiment in FIG. 6;

FIG. 9 is a sectional view of one of the wire structures of the implant in said another configuration of the second embodiment in FIG. 8.

### DETAILED DESCRIPTION OF THE PREFERRED EMBODIMENTS

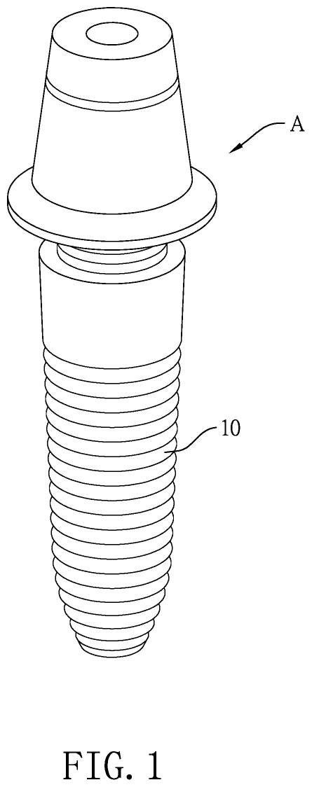

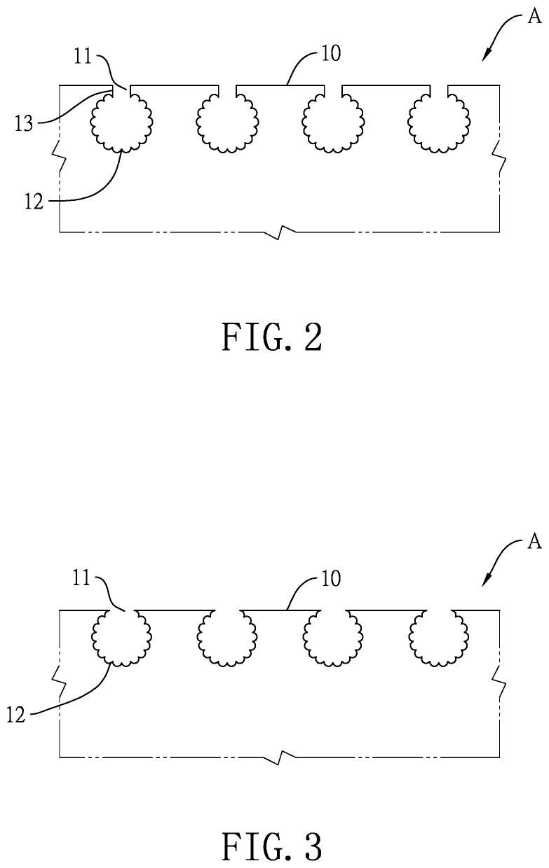

With reference to FIG. 1 and FIG. 2, an implant in accordance with the present invention is provided. In a first embodiment, the implant is an artificial tooth root A for the dental implant surgery and comprises an outer surface 10 and a plurality of cavities 12. The implant selectively further comprises a plurality of passages 13.

The outer surface 10 forms a plurality of openings 11. The cavities 12 respectively communicate with the openings 11. In this embodiment, each one of the cavities 12 communicates with the corresponding opening 11 via a respective one of the passages 13. Precisely, one end of each passage 13 communicates with one of the openings 11 and another end of said passage 13 communicates with one of the cavities 12. In this embodiment, each opening 11 communicates with one passage 13 and one cavity 12; in another embodiment, one opening 11 may communicate with one cavity 12 only. FIG. 2 shows the arrangement of the cavities 12. In FIG. 2, the openings 11 and the cavities 12 are arranged regularly on the outer surface 10; in another embodiment, the openings 11 and the cavities 12 may be arranged irregularly as shown in FIG. 3. In another configuration of this embodiment, the implant may not comprise any passage, such that the cavities 12 communicate with the openings 11 directly.

The longitudinal section of each one of the cavities 12 is perpendicular to the outer surface 10 and may be round, elliptical, oval, or triangular. The latitudinal section of each one of the cavities 12 is parallel with the outer surface 10, and the latitudinal section thereof is changed according to the distance from the outer surface. The maximum area of the latitudinal section is larger than the sectional area of the communicating opening 11. Therefore, the cavities 12 have more spaces to contain medicine. Further, the surface of each one of the cavities 12 may be smooth, or comprise irregular concave and convex structures.

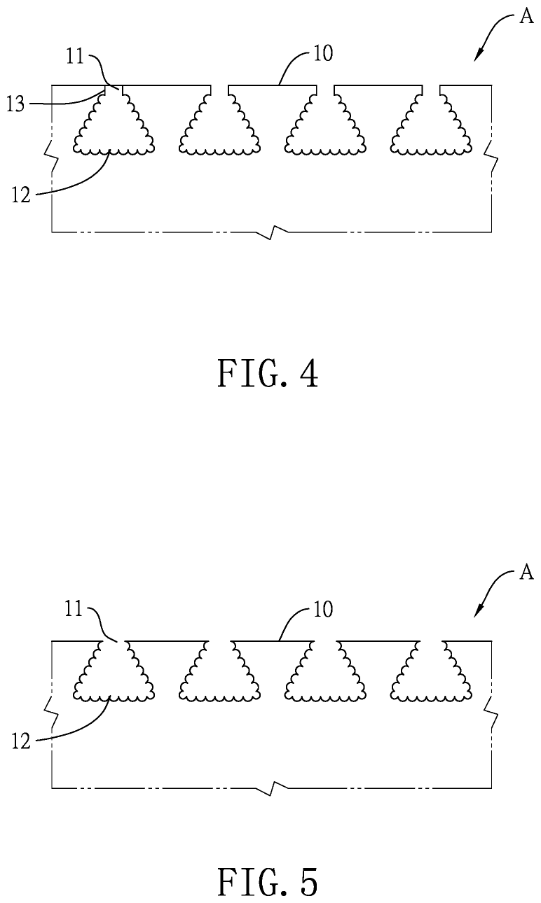

In the configurations as shown in FIG. 4 and FIG. 5, the latitudinal section of each one of the cavities 12 is as larger as farer from the outer surface 10. In other words, in these configurations, the longitudinal sections of the cavities 12 may be triangular.

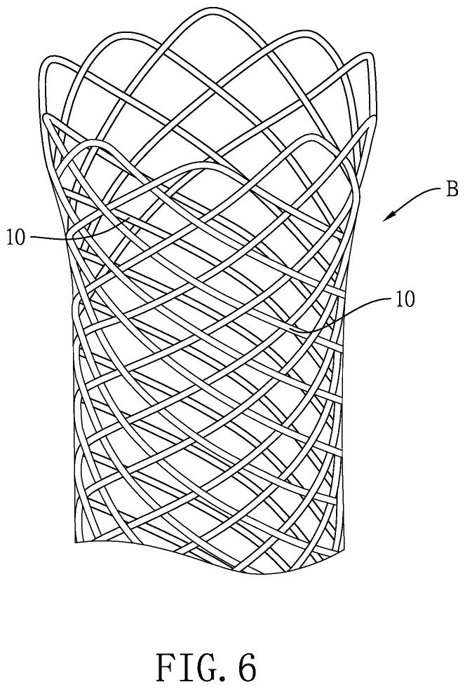

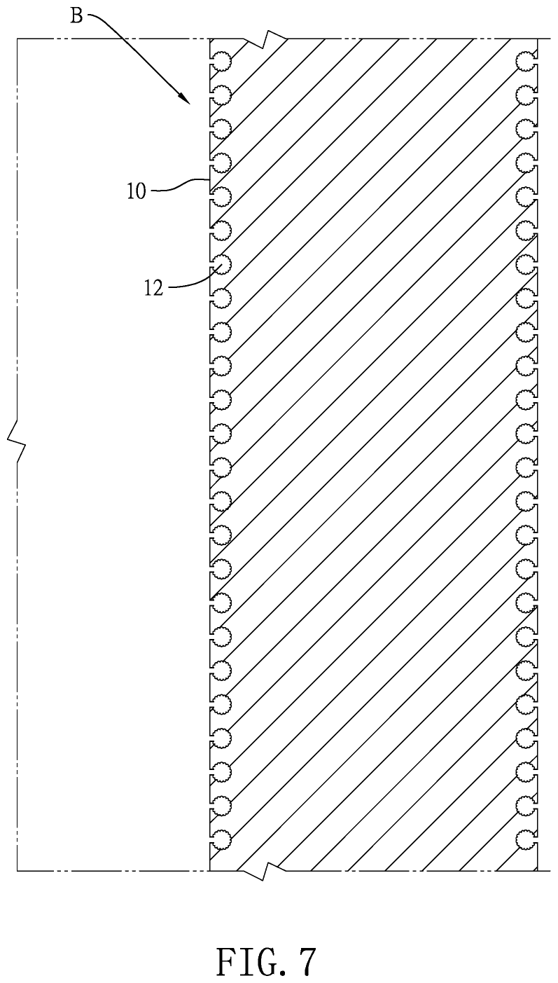

Then please refer to FIG. 6 and FIG. 7. In a second embodiment of the present invention, the implant is a stent B for cardiac catheterization. The stent B is roughly in the shape of a tube, so the outer surface 10 of the stent B includes an outward cylinder surface and an inward cylinder surface. In this embodiment, both of the outward cylinder surface and the inward cylinder surface form the openings 11 and the cavities 12; however, in another embodiment, the openings 11 and the cavities 12 may only be formed on the outward cylinder surface.

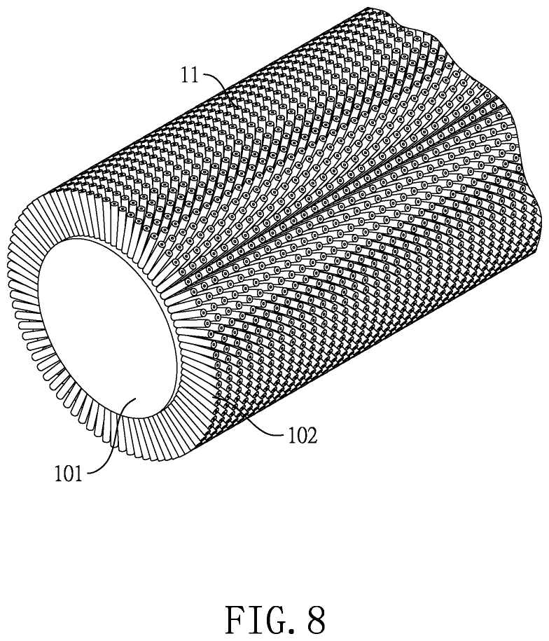

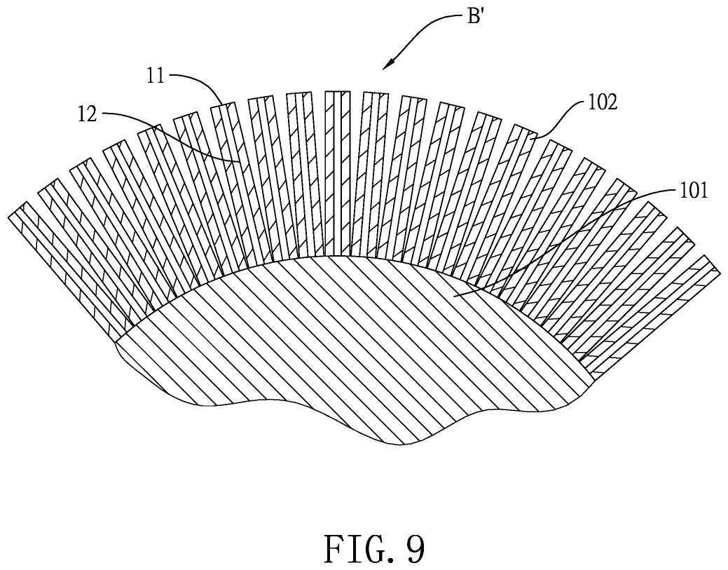

Then please refer to FIG. 8 and FIG. 9, which shows another configuration of the stent B′ in the second embodiment. FIG. 8 shows one of the wires that forms the mesh tube (i.e. the stent B′) and FIG. 9 shows the sectional view of the wire. Each one of the wire structures 101 of the stent B′ forms a plurality of tube structures 102 located thereon and arranged densely. For example, ends of the tube structures 102 that are connected to the wire structure 101 are adjacent to each other. Each one of the tube structures 102 forms a cavity 12 therein. One end of each tube structure 102 is connected to the wire structure 101 and another end forms the aforementioned opening 11. The opening 11 communicates with the cavity 12. Because the tube structures 102 are arranged densely, end surfaces of the ends of the tube structures 102 that are away from the wire structure 101 form the outer surface of the stent B′ together.

With the aforesaid structure, the outer surface of the implant forms multiple openings 11 communicating with the cavities 12, and therefore the medicine can flow into the cavities 12 after the medicine is applied on the surface of the implant. Because the maximum of the latitudinal section of each cavity 12 is larger than the corresponding opening 11, the medicine may not flow out after flowing into the cavity 12, which allows the implant to absorb more medicine. If the implant is an artificial tooth root A, the medicine may include ingredients for enhancing the gum bone growth. Therefore, the implant of the present invention can absorb more medicine that enhances bone growth, which shortens the period for fixing the artificial tooth root A on the gum. On the other hand, if the implant is a stent B, the medicine may include ingredients for preventing angiogenesis. Therefore, the implant of the present invention can absorb more medicine that prevents angiogenesis, which decreases vascular blockage possibility.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An implant comprising:
an outer surface forming a plurality of openings; and
a plurality of cavities communicating with the openings respectively.

2. The implant as claimed in claim 1, wherein a maximum area of a latitudinal section of each one of the cavities is larger than a sectional area of the corresponding opening and the latitudinal sections thereof are parallel with the outer surface.

3. The implant as claimed in claim 1 further comprising a plurality of passages, one end of each one of the passages communicating with a respective one of the openings, and another end of each one of the passages communicating with a respective one of the cavities.

4. The implant as claimed in claim 1, wherein a longitudinal section of each one of the cavities is round, elliptical, oval, or triangular; the longitudinal sections of the cavities are perpendicular to the outer surface.

5. The implant as claimed in claim 3, wherein a longitudinal section of each one of the cavities is round, elliptical, oval, or triangular; the longitudinal sections of the cavities are perpendicular to the outer surface.

6. The implant as claimed in claim 1, wherein a latitudinal section of each one of the cavities is as larger as farer from the outer surface; the latitudinal sections of the cavities are parallel with the outer surface.

7. The implant as claimed in claim 5, wherein the latitudinal section of each one of the cavities is as larger as away from the outer surface; the latitudinal sections of the cavities are parallel with the outer surface.

8. The implant as claimed in claim 1, wherein the implant is an artificial tooth root.

9. The implant as claimed in claim 7, wherein the implant is an artificial tooth root.

10. The implant as claimed in claim 1, wherein the implant is a stent.

11. The implant as claimed in claim 7, wherein the implant is a stent.

12. An implant comprising:
a plurality of wire structures; and
a plurality of tube structures formed on each one of the wire structures; each one of the tube structures forming a cavity, one end of each one of the tube structures connected to the wire structure, and another end forming an opening communicating with the cavity;
wherein the implant is a stent.
